(19) United States
(12) Patent Application Publication
(10) Pub. No.: US 2026/0346048 A1
(43) Pub. Date: Nov. 5, 2026

(54) Brachytherapy Applicators for Cervical Cancer Treatment

(71) Applicant: ICAHN SCHOOL OF MEDICINE AT MOUNT SINAI, New York, NY (US)

(72) Inventors: Megan Christy, New York, NY (US); Nicola Nasser, New York, NY (US); Joseph A. Borrello, New York, NY (US); Iden Kurtaliaj, New York, NY (US)

(73) Assignee: ICAHN SCHOOL OF MEDICINE AT MOUNT SINAI, New York, NY (US)

(21) Appl. No.: 19/647,278

(22) Filed: Apr. 14, 2026

Related U.S. Application Data
(60) Provisional application No. 63/789,091, filed on Apr. 15, 2025.

(51) Int. Cl. A61N 5/10

(52) U.S. Cl. CPC A61N 5/1016; A61N 5/1007; A61N 2005/1008; A61N 2005/1011

(57) ABSTRACT

A brachytherapy applicator for a cervical cancer treatment includes a hollow winged tandem configured for insertion through the cervix. The hollow winged tandem has expandable wings and a movable cover to expose and expand the expandable wings, wherein the hollow winged tandem is configured to remain inside a patient during the entire cervical cancer treatment. A cup member secures the applicator to a base of the cervix. The cup member includes a cup portion at a first end thereof and at least one internal channel, and the cup member includes an upper post partially disposed within the cup portion and extending beyond the first end. The cup member with the post is configured to slide into the hollow winged tandem to allow for insertion of the cup member at a beginning of each patient visit and for removal before the patient is discharged.

### CROSS-REFERENCE TO RELATED APPLICATION

The present application claims priority to and the benefit of US patent application Serial No. 63/789,091, filed Apr. 15, 2025, which is hereby expressly incorporated by reference in its entirety.

### TECHNICAL FIELD

The present application is related to brachytherapy and more particularly, to an applicator for use in a cervical cancer brachytherapy treatment.

### BACKGROUND

Brachytherapy is a type of internal radiation therapy used to treat cancer by placing radioactive (radiation) sources (seeds, ribbons, wires, rods or capsules) directly into or next to a tumor. This method allows for a high, targeted dose of radiation to kill cancer cells while minimizing damage to surrounding healthy tissue. Brachytherapy is a common therapy used to treat cervical cancer, whereby a doctor inserts a specialized applicator (tube) into the vagina or uterus under anesthesia. A machine then passes a radioactive source into the applicator for a short time (minutes) to treat the tumor. There are two types of brachytherapy for cervical cancer, namely, a high-dose rate (HDR), which is a common outpatient procedure where high doses are delivered in short 10-20 minute sessions over several days, and low-dose rate (LDR) in which radiation is delivered continuously over a few days, often requiring a hospital stay.

One of the disadvantages of brachytherapy therapy is that brachytherapy applicators are large and painful, relying on their size for stability within the vagina and making the procedure highly uncomfortable for patients. The stability is essential because the applicator cannot move between imaging and treatment while the physicists and oncologists plan the treatment. Current applicators require the blind assembly of three or more parts inside of the vaginal canal and must be inserted under anesthesia, requiring a high level of expertise and limiting the accessibility of the procedure.

There is therefore a need for a simple applicator that allows dose conformity to the tumor while eliminating the need for anesthesia.

### SUMMARY

In accordance with one embodiment, an applicator and corresponding brachytherapy system are disclosed and are constructed to provide improved fit and stability of the device within a patient’s anatomy. The most important consideration during a brachytherapy procedure is that the position of the applicator does not move from the time of imaging to treatment, because even just millimeters of movement can impact the dose and damage tissue in critical organs and leave cancerous tissue untreated.

The solution advanced in the present disclosure is a comfortable applicator with a thin, single-channel tandem and an expandable, single-channel cup or multi-channel cup. The cup can have multiple channels that extend along the sides of the cup or can include a center longitudinal channel and a single spiral channel in one embodiment. Upon insertion of the device, the oncologist will fold the cup into a U-shape around the tandem. Once in place, the oncologist lets go of the cup and it will naturally return to its original shape and sit within the vaginal canal at the base of the cervix, held in place by suction.

Next, a connection is provided to a High-Dose Rate (HDR) afterloader 200 (FIG. 4) to facilitate a passing of the at least one radioactive source into one or more channels to reach one or more locations within the one or more channels. The placement of the radioactive source in these one or more target location results in an irradiation of at least one portion of the uterus via the at least one radioactive source so as to treat the cervical and/or uterine cancer.

After treatment, the cup is removed; however, the tandem is intended to remain in place. The construction of the applicator thus facilitates the patients to stand, walk and function normally after its insertion, allowing patients to walk to the MRI suite after the insertion of the applicator, and then walk to a the HDR room, and be able to function within normal parameters while the tandem remains in place within the uterus between the brachytherapy treatments

### BRIEF DESCRIPTION OF THE DRAWING FIGURES

FIG. 1 is a side view of an applicator according to a first embodiment inserted into the uterus and held in place therein;

FIG. 2 is a side and top perspective view of the applicator in an assembled state;

FIG. 3 is a side and top perspective view of an applicator including a winged tandem and a spiral channel cup;

FIG. 4 is an exploded perspective view of a brachytherapy system comprising the applicator of FIG. 3 along with an afterloader and transfer tubes;

FIG. 5 is a side perspective view of an applicator comprising a winged tandem and a spider cup;

FIG. 6A is a side perspective view of the spider cup;

FIG. 6B is a side perspective view of the tandem;

FIG. 7 is a side perspective view of a protective tandem sheath;

FIG. 8 is a side perspective view of a protective cup sheath; and

FIG. 9 is a bottom plan view of the spider cup of FIG. 6A.

### DETAILED DESCRIPTION OF CERTAIN EMBODIMENTS

The applicator, system and methods described herein are primarily intended for use in brachytherapy treatment of cervical cancer; however, other potential applications, such as the treatment of uterine cancer, are possible.

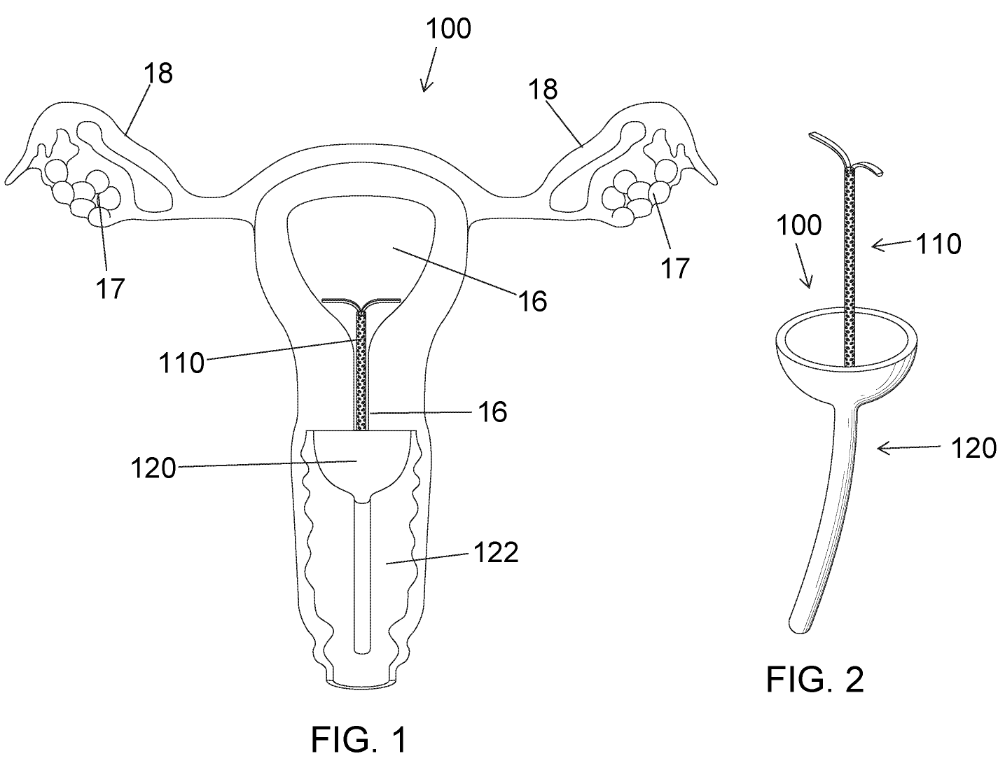

FIG. 1 illustrates the female anatomy and in particular, shows the female reproductive system 10. The female reproductive system 10 includes vagina 12, uterus 14, cervix 16, ovaries 17 and fallopian tubes 18. The vagina 12 is a muscular canal that joins the cervix 16 (the lower part of uterus 14) to the outside of the body. The cervix 16 is the lower, narrow part of the uterus 14 that connects to the vagina 12. The uterus 14 is a hollow, pear-shaped organ that holds a fetus during pregnancy. The uterus 14 is divided into two parts: the cervix 16 and the corpus. The corpus is the larger part of your uterus that expands during pregnancy. The ovaries 17 are small, oval-shaped glands that are located on either side of the uterus 14. The fallopian tubes 18 are narrow tubes that are attached to the upper part of the uterus 14 and serve as pathways for the eggs to travel from the ovaries 17 to the uterus 14.

Cancer can impact either the uterus 14 or the cervix 16. Cancer types are determined by where the cancerous growth begins. If cancer starts in the lining of the uterus 14 (endometrium) or its muscle and connective tissue, the diagnosis is uterine cancer. If cancer begins in the cells of the cervix 16 (the lower end of the uterus 14), the diagnosis is cervical cancer. Developing cervical cancer and uterine cancer at the same time is possible but fairly rare.

There are several traditional treatments for uterine and cervical cancers. For example and with respect to the uterine cancer, treatment options include: 1) surgery: the most common treatment to remove the cancer, usually involving a total hysterectomy; 2) radiation therapy that destroys cancer cells and can include external beam radiation (from a machine outside the body) or brachytherapy (internal radiation placed inside the vagina); and chemotherapy: often used for higher-risk or advanced cancers, typically involving drugs like carboplatin and paclitaxel to kill cancer cells, often administered after surgery.

The applicators and systems and methods described herein relate to brachytherapy and can be used for treatment of uterine cancer and/or cervical cancer as described in detail herein.

### Applicator 100

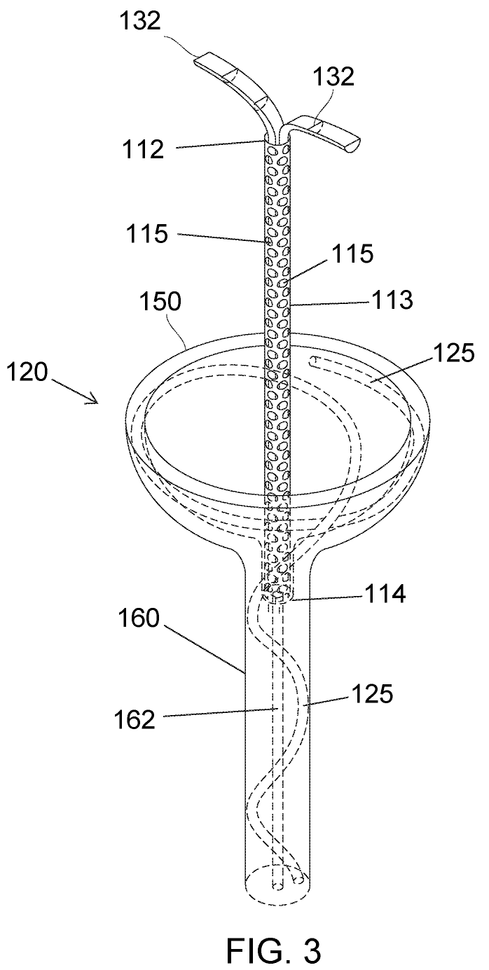

Now referring to FIGS. 1-4, a high-dose rate (HDR) (intrauterine) applicator 100 according to one exemplary embodiment is illustrated. The applicator 100 consists of two separate parts that can be easily separated from one another. More specifically, the applicator 100 comprises a single-channel tandem (which also can be referred to as being a tandem sheath) 110 and a collapsible/expandable (pliable) cup 120 with the tandem 110 can be considered to be an intrauterine device since it is intended for insertion into the uterus 14 and the cup 120 is intended for placement near or at the cervix 16 (e.g., the cup 120 can hug the cervix 16).

### Tandem 110

The tandem 110 comprises an elongated devices that has a first end 112 and an opposite second end 114 and comprises a lumen device in that it has a tubular construction with at least one lumen or channel formed therein. This at least one lumen or channel functions as a radiation delivery channel and permits delivery of a radioactive (radiation) source, such as at least one radioactive seed or radioactive material, to the uterus 14. The length of the tandem 110 is such that the second end 114 extends outside of the uterus 14. At the first end 112, the tandem 110 includes a fixation device 130 or mechanism that is configured to fix the tandem 110 within the uterus 14 in a non-permanent manner. As described in more detail herein, the fixation device 130 is designed to fix the tandem 110 to the uterus 14 without piercing a wall of the uterus 14.

A main elongated body 113 of the tandem 110 is perforated in that it includes through holes 115 along its surface. As shown, the perforations (holes 115) can extend completely from the first end 112 to the second end 113. These perforations assist in delivery of the radioactive source to the target tissue site (uterus 14) since the radioactive source is located within the at least one lumen / channel of the main elongated body 113. In one embodiment, there is a single center lumen / channel (linear channel) that extends the length of the main elongated body 113.

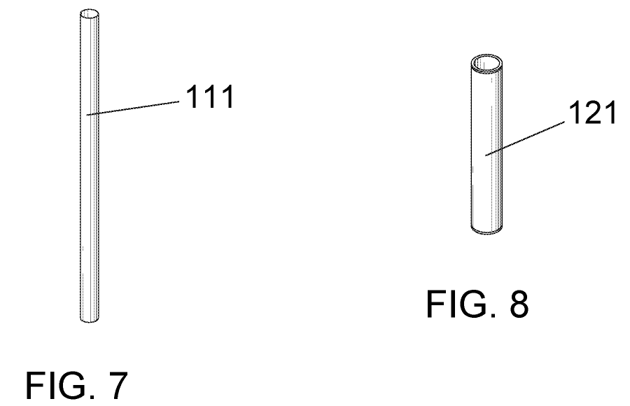

The fixation device 130 located at the second end 113 is constructed to effectively fix or anchor the tandem 110 within the uterus 14, while permitting removal thereof at a later date. To assist in the delivery of the tandem 110 to the uterus 14, the fixation device 130 can be constructed to have a collapsed or retracted state and an expanded state. For example, the fixation device 130 includes a pair of wings 132 that, in the expanded state, extend radially outward from the main elongated body 113. In the expanded state, the wings 132 combined with the main elongated body 113 has a T-shape. To deliver the tandem 110 with the wings 132 in the collapsed state, a tandem sheath 111 (FIG. 7) can be used and is simply fitted over the tandem 110 to force the wings 132 to assume the collapsed state. Once the sheath 111 is removed from the tandem 110 and in particular, once the sheath 111 is removed from covering the wings 132, as by sliding the sheath 111 off the wings 132, the wings 132 automatically deploy to the expanded state. To move automatically from the collapsed state to the expanded state, the wings 132 can be formed of a shape memory material or can be otherwise formed and constructed so that they have shape memory characteristics permitting automatic deployment.

In the illustrated embodiment, the main elongated body 113 of the tandem 100 is a cylindrical shape and each wing 132 can be generally rectangularly shaped. Typically, the perforations are uniformly formed in the body 113; however, other patterns are possible.

The radioactive source delivered through the channel formed within the tandem 110 can be in the form of a wire, seed, rod, etc. As shown in FIG. 1, tandem 110 is partially within the uterus to permit anchoring of the tandem 110 and extends (passes) through the cervix so that the perforated body 113 is at least partially located within the cervix to allow treatment of the cervix by the radioactive source within the hollow tandem 110.

### Cup 120

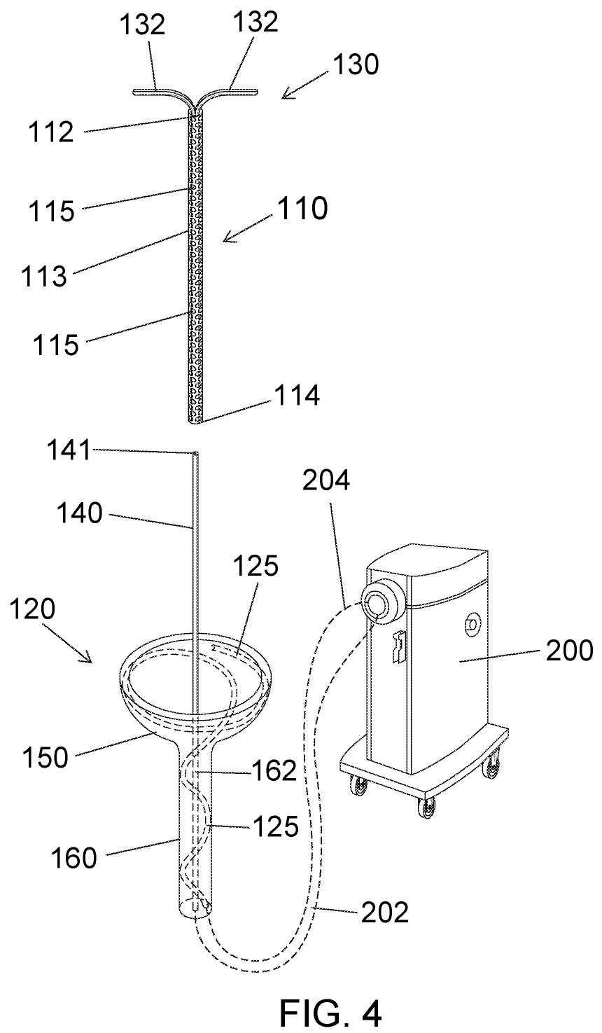

The other part of the applicator 100 is the collapsible/expandable (pliable) cup 120 for placement at or near the cervix. As shown in FIG. 4, the cup 120 has a number of different sections or parts, including, an upper tubular member (upper post or upper tandem) 140, a cup-shaped body 150 and a lower tubular shaft 160. The upper tubular member 140 extends upwardly away from a top open edge 151 of the cup-shaped body 150. The upper tubular member 140 is a hollow (linear) structure and thus includes at least one lumen and preferably has a single center lumen 141. The upper tubular member 140 is intended to act as a coupler for coupling the tandem 110 to the cup 120. More specifically, the tandem 110 is inserted over the upper tubular member 140 due to the fact that the diameter of the upper tubular member 140 is less than the diameter of the tandem 110. By inserting the tandem 110 over the upper tubular member 140, not only are the two structures coupled to one another but also a fluid connection is established between the tandem 110 and the upper tubular member 140. This connection allows the radioactive source to be inserted and delivered through the upper tubular member 140 to the tandem 110. The upper tubular member 140 is therefore made of a material that permits radioactive transmission therethrough.

The upper tubular member 140 can be a separate part from the pliable cup and is inserted into a center lumen or hole or bore formed in the cup to couple the upper tubular member 140 to the cup. This coupling is a secure coupling to allow for insertion and removal of the tandem 110 from the upper tubular member 140 without the disrupting the connection between the upper tubular member 140 and the cup.

The lower tubular shaft 160 includes at least one channel 162 formed therein. This channel 162 is in registration (i.e., coaxial to) with the upper tubular member 140 so that an object, such as the radioactive source, can be inserted into and pass through the channel 162 into the upper tubular member 140, and thus, ultimately, into the inside of the tandem 110. The lower tubular member 160 can be a cylindrical member with the channel 162 being a single central channel formed therein and open at the bottom of the lower tubular shaft 160.

As shown in FIG. 8, the cup 120 includes a cup cover 121 to protect the cup 120 when it is not being used. Due to the pliable nature of the cup 120, the cup cover 121 can be a cylindrical tube that is sized to receive the cup 120 in its collapsed (folded) state.

In contrast to the remaining parts of the cup 120, the upper tubular member 140 is preferably a rigid structure. The cup-shaped body 150 and the lower tubular shaft 160 are flexible (pliable) to allow them to be collapsed and more particularly, the cup-shaped body 150 is capable of being folded so as to substantially reduce its footprint during insertion into the female anatomy (i.e., the vaginal canal). For example, the cup-shaped body 150 can be folded in a U-shape around the tandem 110 similar to how a menstrual cup is folded for insertion. The memory shape properties of the cup-shaped body 150 means that once the applicator 100 is in its target place, the oncologist lets go of the cup-shaped body 150 and it naturally returns to its original shape and sits within the vaginal canal at the base of the cervix.

The pliable nature of the cup-shaped body 150 allows the applicator 100 to have optimal fit and stability within the patient’s anatomy. The most important consideration during a brachytherapy procedure is that the position of the applicator from the time of imaging to treatment, because, even just small movements (i.e., millimeters) can impact the dose and damage tissue in critical organs and leave cancerous tissue untreated. The cup 120 thus primarily permits anchoring of the applicator 100 within the cervix and surrounding areas.

The cup 120 can be thought of as a spiral channel cup due to its internal spiral channel architecture. The spiral nature is due to the fact that the cup 120 has an integral single spiral channel 125 formed therein. As shown, the single spiral channel 125 beings at the open bottom end of the lower tubular shaft 160 and extends in a spiral manner up to the top edge of the cup-shaped body 150. The single spiral channel 125 terminates with a closed end near or at the top edge of the cup-shaped body 150. The single spiral channel 125 is open at the open bottom end of the lower tubular shaft 160 and thus, is capable and intended for receiving a radioactive source for delivering radiation through the cup-shaped body 150. For example, the radioactive source can be fed through the single spiral channel 125 and delivered to the end of the single spiral channel 125. For example, the radioactive source can be in the form of a wire, seed, rod, etc. that can be delivered to and can navigate through the single spiral channel 125.

In one embodiment, the applicator 100 has the following characteristics: cup 120: OD = 9mm, channel ID = 1.5mm (elastic) (Optimal Turn Radius: 4.34 mm); tandem 110 (attached to spiral cup): OD = 2mm, ID = 1.5 mm, height = 70 mm (durable); winged Tandem Sheath: OD = 3mm, ID = 2.3 mm (durable/elastic) (insert Channel OD = 2 mm); tandem cover: OD = 4mm, ID = 3.5mm (durable); and cup Cover: OD = 12.6 mm, ID = 10.6 mm (durable).

One exemplary procedure comprises the following steps: 1) the tandem 110 is inserted through the cervix and then 2) the cover 111 is pulled back to expand the wings 132. The tandem 110 stays inside of the patient during the entire course of their treatment (3-5 weeks). This component (tandem 110 / cover 111) is disposable. An additional step includes: 3) sliding the cup 120 over the tandem 110. The cup 120 is inserted at the beginning of each visit, and then removed before the patient is discharged. This component (cup 120) is sterilized between each use. The smooth cup 120 is intended to be inserted like a menstrual cup, where the cup is folded to minimize diameter on insertion and then released to form back into the cup shape when near the cervix. As described, the spiral cup has a cover that it can be inserted with and then subsequently withdrawn to allow the cup to expand and form around the cervix and be held in place by suction. The cup 120 thus serves to stabilize the entire applicator and hold it in place within the vaginal canal, thereby allowing the tandem 110 to be securely positioned within the cervix.

Next, a connection is provided to a High-Dose Rate (HDR) afterloader 200 (FIG. 4) to facilitate a passing of the at least one radioactive source into one or more channels to reach one or more locations within the one or more channels. The placement of the radioactive source in these one or more target location results in an irradiation of at least one portion of the uterus via the at least one radioactive source so as to treat the cervical and/or uterine cancer. When multiple channels are present, it increases the ability to contour the tumor with radiation dose.

FIG. 4 shows the applicator 100 as part of a brachytherapy treatment system. The system includes an HDR remote afterloader 200 that comprises a shielded machine that stores the radioactive source (e.g., Ir – 192). There is also a drive mechanism (not shown), that is typically controlled by a computer, to move the radioactive source from the safe (afterloader 200) into the applicator 100 through transfer tubes 202, 204. FIG. 4 generally shows that one transfer tube 202 is in communication with the channel 162 of the lower tubular shaft 160 and the other transfer tube 204 is in communication with the single spiral channel 125.

In one embodiment, the radiation source—often a single tiny, high-intensity radioactive pellet (often Iridium-192)—is attached to a motorized cable that is pushed out of the afterloader 200 and into one of the transfer tubes.

### Multi-Channel Cup 300

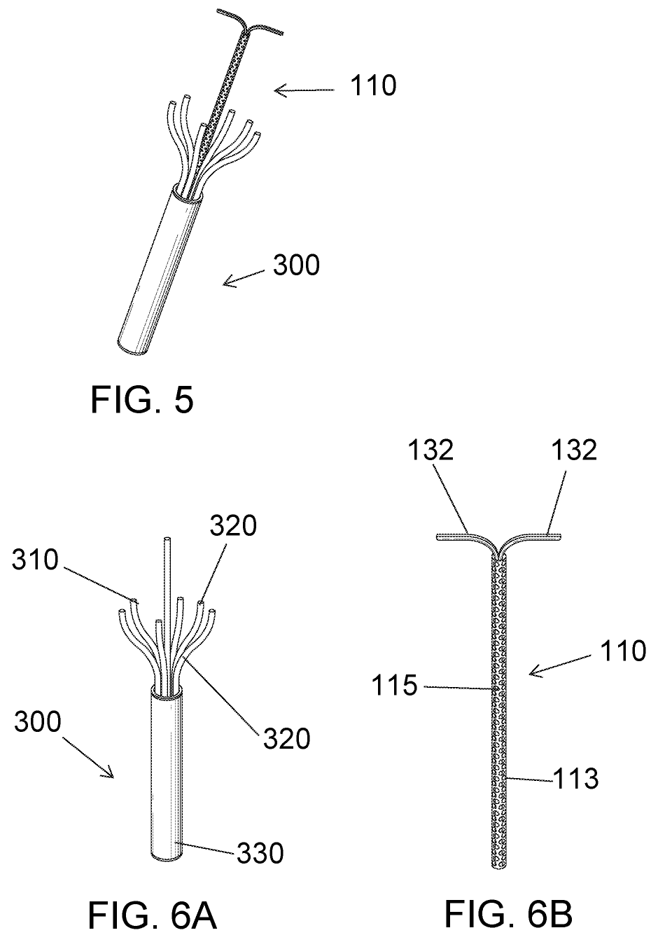

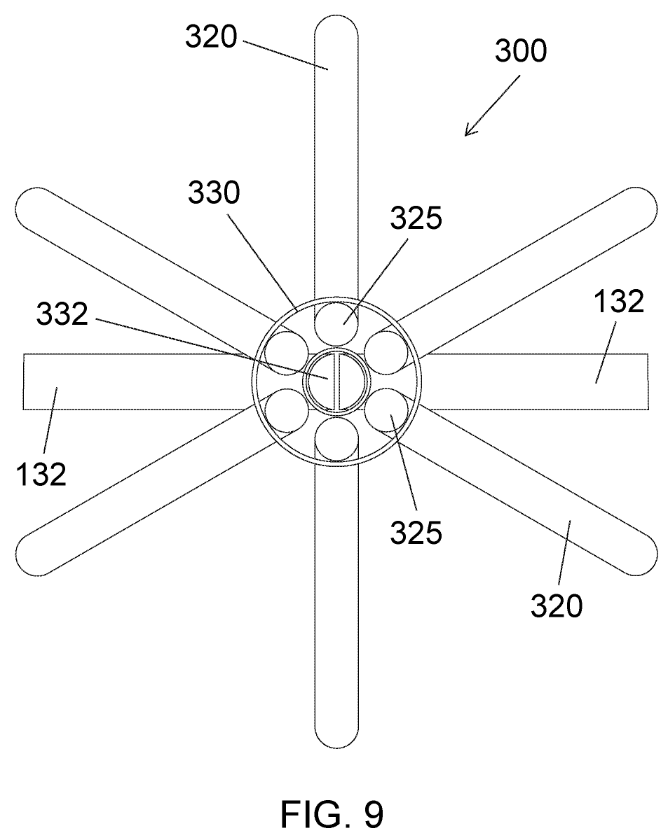

Now referring to FIGS. 5, 6A, 6B, and 9, , in which an alternative collapsible/expandable (pliable) cup 300 for placement at or near the cervix is illustrated. Due to its construction, as described below, this cup 300 can be referred to as being a spider cup. As shown in FIG. 6A, the cup 300 has a number of different sections, including, an upper tubular member 310, a plurality of conduit members 320 and a lower tubular shaft 330. The upper tubular member 310 can be the same or similar to the upper tubular member 140 and extends upwardly above the other sections. The upper tubular member 310 is a hollow (linear) structure and thus includes at least one lumen and preferably has a single center lumen 311. The upper tubular member 310 is intended to act as a coupler for coupling the tandem 110 to the cup 300. More specifically, the tandem 110 is inserted over the upper tubular member 310 due to the fact that the diameter of the upper tubular member 310 is less than the diameter of the tandem 110. By inserting the tandem 110 over the upper tubular member 310, not only are the two structures coupled to one another but also a fluid connection is established between the tandem 110 and the upper tubular member 310. This connection allows the radioactive source to be inserted and delivered through the upper tubular member 310 to the tandem 110. The upper tubular member 310 is therefore made of a material that permits radioactive transmission therethrough.

The lower tubular shaft 330 includes at least one channel 332 formed therein. This channel 332 is in registration (i.e., coaxial to) with the upper tubular member 310 so that an object, such as the radioactive source, can be inserted into and pass through the channel 332 into lumen 311 of the upper tubular member 310, and thus, ultimately, into the inside of the tandem 110. The lower tubular member 330 can be a cylindrical member with the channel 332 being a single central channel formed therein and open at the bottom of the lower tubular shaft 330.

Unlike the cup 120 which has a single cup-shaped body, the cup 300 has a plurality of conduit members 320 each of which has a dedicated internal channel formed within the lower tubular shaft 330. The plurality of conduit members 320 take the form of a plurality of pliable fingers that extend above the top edge of the lower tubular shaft 330. Like the cup-shaped body in the previous embodiment, each conduit member 320 is capable of moving between a collapsed or retracted state and a deployed or expanded state. When the cup sheath 121 is slid over the plurality of conduit members 320, they are forced into the retracted states. Once the sheath 121 is removed from the plurality of conduit members 320 automatically spring back to their original expanded states allowing the spider cup 300 to expand and form around the cervix.

The plurality of conduit members 320 are located radially around the upper tubular member 310. Preferably, as illustrated, the plurality of conduit members 320 provide 360 degree of coverage and are circumferentially spaced apart from one another. In the illustrated embodiment, the free ends of the conduit members 320 can all face upward and therefore, in some embodiments, each conduit member 320 can generally have an S-shape given by the fact that the conduit member first extends outwardly from the top of the lower tubular member and then curve and extend upwardly.

As described, each conduit member 320 has a dedicated (feed) channel formed internally within the lower tubular shaft 330 to allow one or more of the conduit members 320 to receive the radioactive source. For example, when radioactive source is associated with a wire or the like, the wire is fed into one select channel of the plurality of dedicated channels resulting in the radioactive source being delivered to a location at or near the cervix. The presence of the radioactive source in one or more of the plurality of conduit members 320 permits brachytherapy of target tissue at a location at or near the cervix.

As shown at the bottom end of the lower tubular shaft 330, there are multiple openings, such as the opening of the center channel 332 and openings for each of the dedicate channels associated with the plurality of conduit members 320. When there are six conduit members 320, there are six peripheral channels 325 that surround the center channel 332 opening and are formed circumferentially therearound. Select or all of the six conduit members 320 can be used to deliver the radioactive source.

Since the conduit members 320 are pliable and since they naturally want to assume the expanded states, they are intended to contact tissue near or at the cervix. The memory properties of the conduit members 320 in effect result in the conduit members 320 pressing against the tissue.

In one embodiment, the components have the following measurements: tandem cover 111: OD = 4mm, ID = 3.3mm (durable); tandem 110: OD = 1.5mm, ID = 2.7mm (durable & flexible/elastic); cup cover 121: OD = 11.9mm, ID = 9.9mm (durable) and cups 120: OD = 8.3mm, channel ID = 1.5mm, tandem ID = 3.3mm (elastic).

In this spider cup embodiment shown in FIGS. 5, 6A, 6B, and 9, each of at least one channel formed in the tandem 110 and the dedicated channels formed in the conduit members 320 comprises a high dose rate (HDR) delivery channel for treating select target tissue areas. In one exemplary embodiment, the disclosed applicator or system can be inserted bedside. After the insertion, a cover that houses the wings can be removed, resulting in the opening of the wings, and attaching the fixation device to the uterus. The HDR channel can exit through the cervix and stay in the vagina. The cup can then be inserted by the physician or the patient, so that the cup is near or hugs the cervix. The vagina can then packed with wet pads or air balloons to increase the space between the cervix, and the rectum and bladder. Thereafter, imaging can be performed with MRI (preferably) or CT scan. Then, the target volumes are drawn, as well as organs at risk. A brachytherapy plan is then generated. After that, the patient can be taken to the HDR room, the intrauterine HDR channel and cup HDR channel(s) can be connected to the HDR afterloader, and the treatment delivered. With HDR, a remote afterloading technology allows a small iridium source attached to the end of a cable to be robotically driven through multiple channels, stopping at predetermined points (dwell positions) for varied lengths of time.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed:

1. A brachytherapy applicator for cervical cancer treatment comprising:
a hollow winged tandem configured for insertion through the cervix, wherein the hollow winged tandem has expandable wings and a movable cover to expose and expand the expandable wings, wherein the hollow winged tandem is configured to remain inside a patient during the entire cervical cancer treatment; and
a cup member for securing the applicator to a base of the cervix, the cup member including a cup portion at a first end thereof and at least one internal channel, the cup member including an upper post partially disposed within the cup portion and extending beyond the first end, wherein the cup member with the post is configured to slide into the hollow winged tandem to allow for insertion of the cup member at a beginning of each patient visit and for removal before the patient is discharged.

2. The brachytherapy applicator of claim 1, wherein the winged tandem is perforated.

3. The brachytherapy applicator of claim 1, wherein the upper post is located partially and centrally inside the cup portion and extends external to the cup portion.

4. The brachytherapy applicator of claim 3, wherein a greater length of the upper post is external to the cup portion than a length internal to the cup.

5. The brachytherapy applicator of claim 4, wherein greater than 50% of a length of the upper post is external to the cup portion.

6. The brachytherapy applicator of claim 1, wherein the at least one channel comprises a plurality of channels.

7. The brachytherapy applicator of claim 1, wherein the at least one channel comprises a single spiral channel.

8. The brachytherapy applicator of claim 1, further including a tandem cover that when pulled back exposes the expandable wings resulting in expansion thereof.

9. The brachytherapy applicator of claim 1, further including a cover for the cup member.

10. The brachytherapy applicator of claim 1, wherein the hollow winged tandem has a pair of expandable wings located 180 degrees apart.

11. The brachytherapy applicator of claim 1, wherein at least the cup portion of the cup is flexible and foldable, while the upper post is rigid.

12. The brachytherapy applicator of claim 1, wherein the at least one channel comprises a central channel that is axially aligned with the tandem and a single spiral channel that extends around the central channel.

13. The brachytherapy applicator of claim 12, wherein the cup member comprises a tubular base and the cup portion is at one end of the tubular base, the single spiral channel extending along the tubular base and the cup portion.

14. The brachytherapy applicator of claim 13, wherein the single spiral channel extends from one end to an opposite end of the cup member.

15. The brachytherapy applicator of claim 12, wherein the upper post is hollow and the central channel is axially aligned and in fluid communication with a central lumen of the upper post.

16. The brachytherapy applicator of claim 13, wherein the tubular base comprises an elongated structure.

17. A brachytherapy applicator for cervical cancer treatment comprising:
a hollow winged tandem configured for insertion through the cervix, wherein the hollow winged tandem has expandable wings and a movable cover to expose and expand the expandable wings, wherein the hollow winged tandem is configured to remain inside a patient during the entire cervical cancer treatment; and
a cup member for securing the applicator to a base of the cervix, the cup member including a plurality of pliable conduit members at a first end thereof and at least one internal channel, the cup member including an upper post partially disposed within the cup portion and extending beyond the first end, wherein the cup member with the post is configured to slide into the hollow winged tandem to allow for insertion of the cup member at a beginning of each patient visit and for removal before the patient is discharged.

18. The brachytherapy applicator of claim 1, wherein each conduit member has a dedicated internal channel.
